(19) United States
(12) Patent Application Publication
(10) Pub. No.: US 2019/0058841 A1
(43) Pub. Date: Feb. 21, 2019

(54) ENDOSCOPE

(71) Applicant: OLYMPUS CORPORATION, Tokyo (JP)

(72) Inventors: Shinji YAMASHITA, Tachikawa-shi (JP); Yuzuru TANABE, Niiza-shi (JP); Yasunori MATSUI, Hino-shi (JP); Yuta MATSUNO, Hachioji-shi (JP)

(73) Assignee: OLYMPUS CORPORATION, Tokyo (JP)

(21) Appl. No.: 16/166,226

(22) Filed: Oct. 22, 2018

Related U.S. Application Data
(63) Continuation of application No. PCT/JP2017/012926, filed on Mar. 29, 2017.

(30) Foreign Application Priority Data
Jun. 29, 2016 (JP) 2016-128781

(51) Int. Cl. H04N 5/367; A61B 1/045; H04N 5/365; H04N 9/73; A61B 1/06; A61B 1/00

(52) U.S. Cl. CPC H04N 5/367; A61B 1/045; H04N 5/3651; A61B 1/00009; A61B 1/0638; A61B 1/00006; H04N 9/735

(57) ABSTRACT

An endoscope connected to a control apparatus, includes an imager, a status signal acquisition circuit, and a preprocessor. The imager images a subject to generate an imaging signal related to the subject. The status signal acquisition circuit receives a status signal indicating an operation status or an operation mode of the endoscope or of the control apparatus. The preprocessor processes the imaging signal according to the status signal received via the status signal acquisition circuit.

### CROSS-REFERENCE TO RELATED APPLICATIONS

This application is a Continuation Application of PCT Application No. PCT/JP2017/012926, filed Mar. 29, 2017 and based upon and claiming the benefit of priority from the prior Japanese Patent Application No. 2016-128781, filed Jun. 29, 2016, the entire contents of both of which are incorporated herein by reference.

### BACKGROUND OF THE INVENTION

### 1. Field of the Invention

The present invention relates to an endoscope connected to a control apparatus.

### 2. Description of the Related Art

In endoscope systems, various types of endoscopes (scopes) for intended uses are connected to a control apparatus that has a processor including an image processing function. In this processor, an image is processed according to the type of connected endoscopes. The processed image is displayed on, for example, a monitor (see, e.g., Jpn. Pat. Appin. KOKAI Publication No. 2007-185349).

### BRIEF SUMMARY OF THE INVENTION

An endoscope according to an aspect of the invention, comprises: an imager configured to image a subject to generate an imaging signal related to the subject; a status signal acquisition circuit configured to receive a status signal indicating an operation status or an operation mode of the endoscope or of the control apparatus; and a preprocessor configured to process the imaging signal according to the status signal received via the status signal acquisition circuit.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing a configuration of an endoscope system including an endoscope according to an embodiment of the present invention.

FIG. 2 is a flowchart explaining a first example of the operation of the endoscope.

FIG. 3 is a flowchart explaining a second example of the operation of the endoscope.

### DETAILED DESCRIPTION OF THE INVENTION

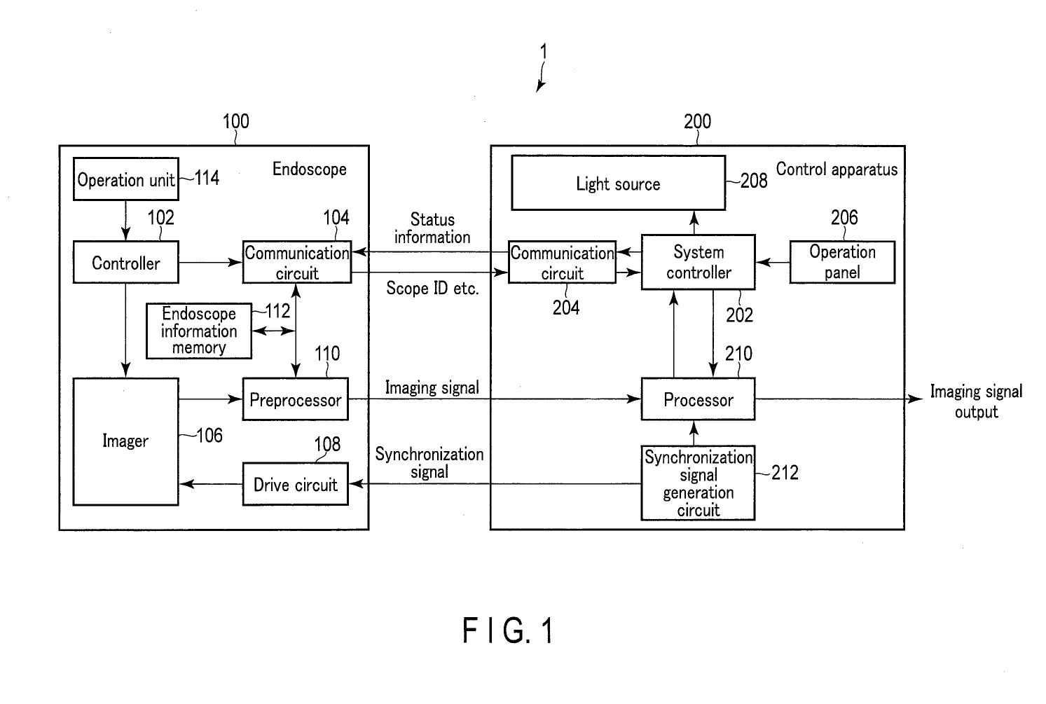

Hereinafter, an embodiment of the present invention will be described with reference to the drawings. FIG. 1 is a diagram showing a configuration of the endoscope system including the endoscope according to the embodiment of the present invention. An endoscope system 1 shown in FIG. 1 has an endoscope (scope) 100 and a control apparatus 200. The endoscope 100 is connected to the control apparatus 200. When the endoscope 100 is connected to the control apparatus 200, the endoscope 100 and the control apparatus 200 can communicate with each other. Communication between the endoscope 100 and the control apparatus 200 is performed by, for example, wired communication via a universal cable. However, the communication between the endoscope 100 and the control apparatus 200 does not necessarily have to be wired.

The endoscope 100 comprises a controller 102, a communication circuit 104, an imager 106, a drive circuit 108, a preprocessor 110, an endoscope information memory 112, and an operation unit 114.

The controller 102 is a control circuit such as a CPU, an ASIC, or an FPGA. It controls each part of the endoscope 100 such as the communication circuit 104 and the imager 106 of the endoscope 100.

As an example status signal acquisition circuit, the communication circuit 104 mediates the communication between the endoscope 100 and the control apparatus 200 under the control of the controller 102 when the endoscope 100 is connected to the control apparatus 200. For example, the communication circuit 104 transfers a status signal transmitted from the system controller 202 of the control apparatus 200 to the endoscope information memory 112. This status signal represents the operation status or operation mode of the endoscope 100 or control apparatus 200. Details of the status signal will be described further below. The communication circuit 104 transmits various kinds of information stored in the endoscope information memory 112 to the processor 210 of the control apparatus 200.

The imager 106 is disposed at the far distal end of the insertion part which is the portion to be inserted into the subject to be examined by the endoscope 100. The imager 106 is a CMOS image sensor or a CCD image sensor. The imager 106 has, for example, a Bayer array color filter. The imager 106 captures the inside of the body of the subject in synchronization with a drive clock from the drive circuit 108, and generates an imaging signal related to the subject.

The drive circuit 108 generates a drive clock synchronized with a synchronization signal transmitted from a synchronization signal generation circuit 212 of the control apparatus 200. The drive circuit 108 then inputs the drive clock to the imager 106. Under the control of the controller 102, the imager 106 performs an imaging operation in synchronization with the drive clock.

The preprocessor 110 performs preprocessing on the imaging signal output as a result of the imaging operation of the imager 106. The preprocessing includes amplification processing of the imaging signal, A/D conversion, pixel interpolation (demosaicing), defective pixel correction, black level correction etc.

The demosaicing is a process of generating, from an imaging signal in which each pixel corresponds to one color component, like a Bayer array, an imaging signal in which each pixel corresponds to a plurality of color components. The preprocessor 110 in the present embodiment is configured to be able to perform different kinds of demosaicing processes, and the demosaicing to be used is appropriately selected according to the status signal inputted from the processor 210. The preprocessor 110, for example, performs demosaicing using either linear interpolation or Adaptive Color Plane Interpolation (ACPI). Linear interpolation is a process of interpolating imaging signals of other color components of a pixel to be interpolated by using an average value of a plurality of imaging signals in the vicinity of a pixel to be interpolated. ACPI is the interpolation of imaging signals of other color components of a pixel to be interpolated using a value obtained by further adding a high frequency component to the linear interpolation result of the interpolation target pixel.

The defective pixel correction includes correcting white defective pixels of the imager 106. A white defective pixel is a pixel in which an imaging signal with higher luminance than that of the imaging signal to be originally output is output by superimposing an excessive dark current component on the imaging signal. The white defective pixel correction is performed by, for example, replacing the imaging signal of the white defective pixel specified in advance at the time of manufacture of the endoscope 100 with the linear interpolation value of the surrounding pixels of the same color. The white defective pixel increases or decreases due to temperature change or deterioration over time. Therefore, in the present embodiment, the position of the white defective pixel is also detected at a specific timing recognized according to the status signal from the processor 210. This timing is, for example, after both the white balance gain acquisition and the turning off of the light source 208 are completed. Details will be described further below. The defective pixel correction may also include correcting black defective pixels of the imager 106. A black defective pixel is a pixel to which an imaging signal is not output.

The black level correction is a process of correcting black level fluctuations (so-called black floating, black sinking) of the imaging signal due to a difference between the black level of the effective pixel area of the imager 106 and the black level of the optical black area of the imager 106. In the present embodiment, the black level correction is performed at a specific timing recognized according to the status signal from the processor 210. This timing is similar to the detection timing of white defective pixels, namely after both the white balance gain acquisition and the turning off of the light source 208 are completed. Details will be described further below.

The endoscope information memory 112 is, for example, a nonvolatile memory and it stores a scope ID which is information for specifying the type of the endoscope 100. The endoscope information memory 112 further stores various parameters such as parameters used for the pre-processing in the endoscope 100 and parameters used for the image processing in the processor 210. Parameters used in preprocessing, for example, include: parameters used for demosaicing such as color filter types and array information; parameters used for defective pixel correction such as positional information about white defective pixels and black defective pixels; and parameters used for black level correction such as the reference black level. The parameters used for the image processing in the processor 210 also include the white balance gain. The endoscope information memory 112 further stores a status signal transmitted from the system controller 202 in the control apparatus 200 via the communication circuit 104. The endoscope information memory 112 is not necessarily a single memory, but may be a plurality of memories. For example, the memory for storing the status signal may be a volatile memory instead of a nonvolatile memory.

The operation unit 114 is disposed in the endoscope 100 and includes operation members for the user to perform various operations of the endoscope 100. These operation members include a knob for bending the endoscope 100 and various operation buttons.

The control apparatus 200 has a system controller 202, a communication circuit 204, an operation panel 206, a light source 208, a processor 210, and a synchronization signal generation circuit 212.

The system controller 202 is a control circuit such as a CPU, an ASIC, or an FPGA. In response to a user's operation of the operation panel 206, the system controller 202 controls the operation of each part of the control apparatus 200 such as the communication circuit 204 and the light source 208 of the control apparatus 200. When the operation mode such as the imaging mode of the endoscope system 1 is changed or when the operation status of the control apparatus 200 changes, the system controller 202 generates a status signal and transmits the generated status signal via the circuit 204 to the endoscope 100.

When the endoscope 100 is connected to the control apparatus 200, the communication circuit 204 mediates communication between the control apparatus 200 and the endoscope 100 under the control of the system controller 202. The communication circuit 204, for example, transmits a status signal to the endoscope 100. The communication circuit 204 further transfers various types of information transmitted from the endoscope 100 to the system controller 202.

The operation panel 206 is a panel comprising various operation members for the user to operate the control apparatus 200. These operation members include operation members such as a switch and a button, and a touch panel. For example, various settings are performed by the operation panel 206, such as settings of operation modes such as an imaging mode. In response to the operation of the operation panel 206, the system controller 202 starts controlling in response to a corresponding operation content. Similarly, in response to the operation of the operation panel 206, the system controller 202 generates a status signal in response to the corresponding operation content.

The light source 208 emits, under the control of the system controller 202, an illuminating light to illuminate the subject. The illuminating light emitted from the light source 208 is transmitted to the endoscope 100 via a light guide (not shown). The illuminating light transmitted to the endoscope 100 is irradiated toward the subject from the tip of the insertion part. The light source 208 in the present embodiment is configured to emit white light or a special light. White light is a light having characteristics of broad intensity with respect to wavelength in the visible wavelength region.

White light is used, for example, to brighten a subject. The special light is a spectral light having a peak near a specific wavelength. The special light is used for highlighted imaging of a specific portion of the subject, such as Narrow-Band Imaging (NBI), Auto-Fluorescence Imaging (AFI), and Infra-Red Imaging (IRI).

The processor 210 performs image processing on the imaging signal pre-processed by the preprocessor 110 to generate image data used for imaging, for example, on a monitor. The image processing performed by the processor 210 includes, for example, white balance correction and gradation correction. The processor 210 then outputs the generated image data to, for example, a monitor. When image data is output to the monitor, an image of the subject imaged by the endoscope 100 is displayed on the monitor.

The synchronization signal generation circuit 212 generates a synchronization signal and transmits the generated synchronization signal to the processor 210 and the drive circuit 108. As a result, the imaging operation of the imager 106 and the image processing of the processor 210 are synchronized.

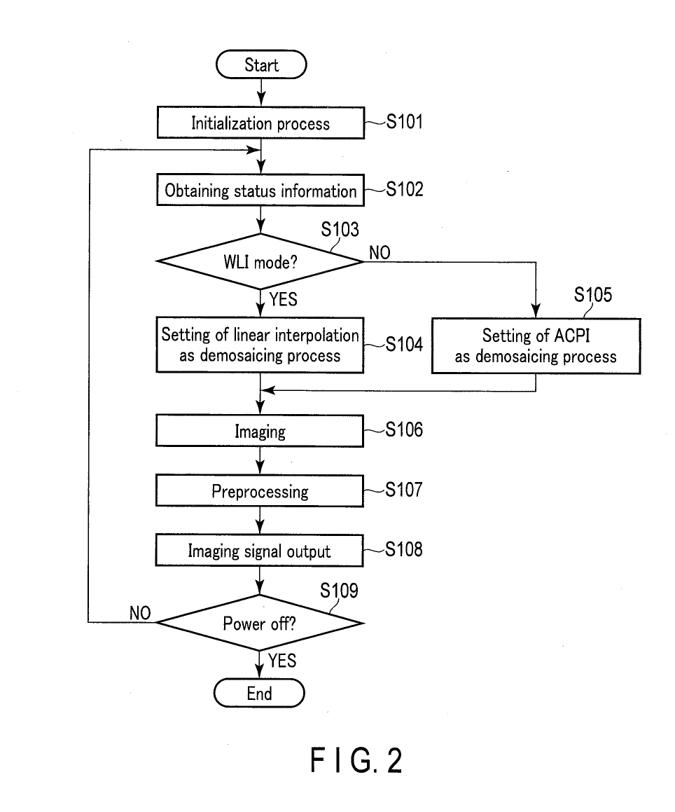

Hereinafter, the operation of the endoscope 100 in the present embodiment will be described. FIG. 2 is a flowchart describing a first example of the operation of the endoscope 100. In the first example, the preprocessor 110 of the endoscope 100 performs different demosaicing processes according to the imaging mode of the endoscope system 1. In the example of FIG. 2, the endoscope system 1 operates in two imaging modes: a white light imaging (WLI) mode and a special light imaging mode. The WLI mode is a mode for observing the subject by irradiating the subject with white light. The special light imaging mode is a mode for observing the subject with any one of Narrow-Band Imaging (NBI), Auto-Fluorescence Imaging (AFI), and Infra-Red Imaging (IRI). The imaging mode is set according to the operation of the operation panel 206 by the user. When the imaging mode is set, the system controller 202 of the control apparatus 200 generates status information indicating the current imaging mode. The system controller 202 then transmits the generated status signal to the endoscope 100. The status signal received by the endoscope 100 is stored in the endoscope information memory 112. The status signal stored in the endoscope information memory 112 is sequentially updated. Status information indicating operation modes such as an imaging mode and status information indicating operation statuses may be stored in different storage areas of the endoscope information memory 112.

When, for example, the endoscope 100 is mounted to the control apparatus 200, the process in FIG. 2 is started. As the endoscope 100 is mounted to the control apparatus 200, the scope ID and various parameters are transmitted from the endoscope 100 to the control apparatus 200. This makes it possible for the processor to perform a processing according to the type of the endoscope 100.

In step S101, the preprocessor 110 initializes the parameters for the preprocessing. In step S101, for example, the gain of the imaging signal and the setting of the demosaicing process executed by the preprocessor 110 are initialized.

In step S102, the preprocessor 110 acquires the status information stored in the endoscope information memory 112. In step S103, the preprocessor 110 refers to the status information and determines whether or not the current imaging mode is the WLI mode. When it is determined in step S103 that the current imaging mode is the WLI mode, the procedure continues to step S104. When it is determined in step S103 that the current imaging mode is not the WLI mode but the special light imaging mode (NBI mode, AFI mode, or IRI mode), the procedure continues to step S105.

In step S104, the preprocessor 110 sets linear interpolation as the demosaicing process. In step S105, the preprocessor 110 sets ACPI as the demosaicing process. After step S104 or step S105, the preprocessor 110 notifies the controller 102 that the setting of the demosaicing process is completed.

In step S106, the controller 102 executes the imaging operation of the imager 106. In step S107, the preprocessor 110 performs preprocessing onto the imaging signal outputted from the imager 106. The preprocessing includes processes such as amplifying the imaging signal from the imager 106, A/D conversion, demosaicing etc. In the demosaicing process, the preprocessor 110 performs demosaicing according to the setting in step S104 or step S105. When, for example, the imaging mode is the WLI mode, linear interpolation is set as the demosaicing process in step S104. In this case, the preprocessor 110 performs linear interpolation onto the imaging signal according to the type and arrangement information of the color filter stored in the endoscope information memory 112. When, on the other hand, the imaging mode is not the WLI mode, i.e., the special light imaging mode, ACPI is set as the demosaicing process of step S105. In this case, the preprocessor 110 performs ACPI onto the imaging signal according to the type and arrangement information of the color filter stored in the endoscope information memory 112. Since images can be obtained in ACPI sharper than in linear interpolation, resolution can be kept high, especially at the edge portion, by selecting ACPI in the special light imaging mode. In the WLI mode, on the other hand, such sharpness is not required at the edge portion. It is therefore possible in the WLI mode to reduce the processing load by selecting linear interpolation. After completing the various preprocesses including the demosaicing process, the procedure continues to step S108.

In step S108, the preprocessor 110 transmits the pre-processed imaging signal to the control apparatus 200 via the communication circuit 104. Upon receiving the imaging signal via the communication circuit 204, the processor 210 performs image processing onto the imaging signal according to the type of the endoscope 100 received in advance. The processor 210 then outputs the image data generated by the image processing to, for example, the monitor.

In step S109, the controller 102 decides whether or not to end the operation of the endoscope 100. When, for example, the endoscope 100 is removed from the control apparatus 200 or when receiving an instruction from the control apparatus 200 to end the operation of the endoscope 100 due to a power-off operation or the like, it is determined that the operation of the endoscope 100 be ended. If it is determined in step S109 that the operation of the endoscope 100 not be ended, the procedure continues to step S102. If it is determined in step S109 that the operation of the endoscope 100 be ended, the process in FIG. 2 ends.

As described above, in the example of FIG. 2, the preprocessor 110 changes the type of demosaicing process to the imaging signal according to the imaging mode indicated by the status signal. In this manner, it is possible, at the endoscope 100, to use the demosaicing process capable of obtaining high resolution imaging signals when the imaging mode requires high resolution, and to lower the processing load when the imaging mode does not require high resolution. In other words, it is not necessary to configure the processor 210 so as to be able to perform a plurality of demosaicing process types, and thus an increase in circuit scale of the processor 210 can be avoided.

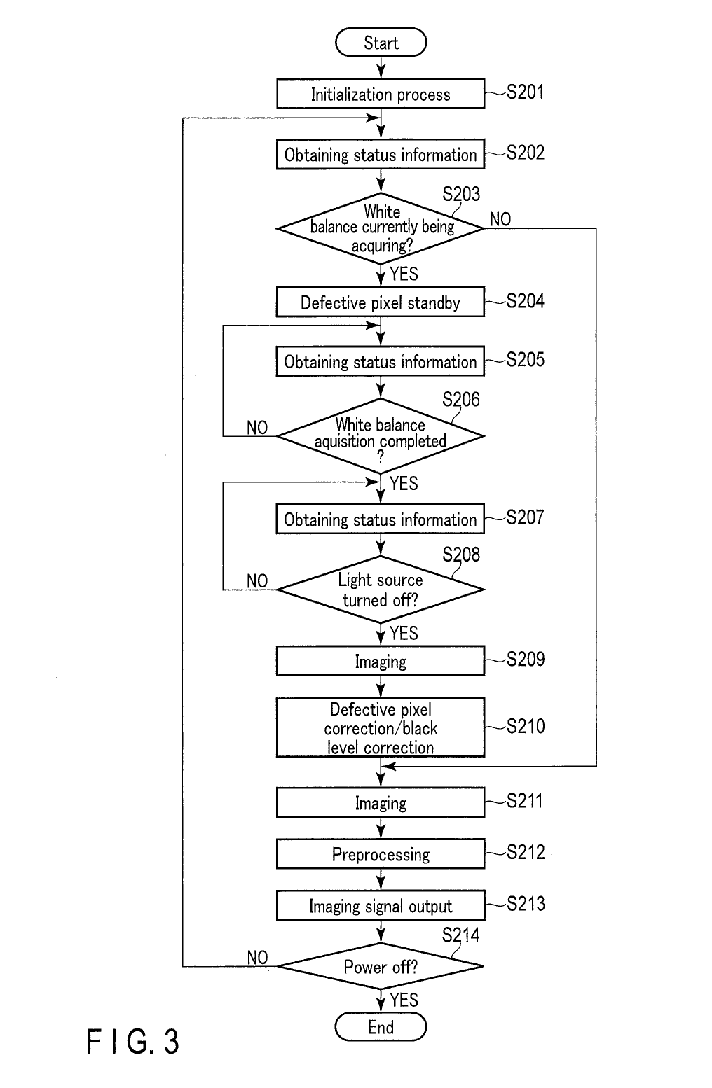

FIG. 3 is a flowchart explaining a second example of the operation of the endoscope 100. In the second example, the preprocessor 110 of the endoscope system 1 performs calibration of defective pixel correction and calibration of black level correction at an appropriate timing. This timing is, as mentioned earlier, after the completion of the white balance adjustment and after turning off the light source.

The process of FIG. 3, for example, is started when the endoscope 100 is mounted on the control apparatus 200. As the endoscope 100 is mounted on the control apparatus 200, the scope ID and various parameters are transmitted from the endoscope 100 to the control apparatus 200. This makes it possible for the processor to perform processing according to the type of the endoscope 100.

In step S201, the preprocessor 110 initializes preprocessing parameters. In step S201, for example, the gain of the imaging signal is initialized.

In step S202, the preprocessor 110 acquires status information stored in the endoscope information memory 112. In step S203, the preprocessor 110 refers to the status information and determines whether or not the current operation status of the control apparatus 200 is currently acquiring white balance. The endoscope system 1 of the example in FIG. 3 has a white balance adjustment function. When using this white balance adjustment function, the user puts a cap called a white balance cap in the insertion part. The user then operates the operation panel 206 to set the operation mode of the endoscope system 1 to the white balance adjustment mode. This starts the white balance adjustment. When, in this embodiment, the operation mode of the endoscope system 1 enters the white balance adjustment mode, the system controller 202 turns on the light source 208. The system controller 202 then transmits a status signal to the endoscope 100, the signal indicating that white balance is currently being acquired. The preprocessor 110 makes the determination of step S203 based on the status signal. If the current operation status of the control apparatus 200 in step S203 is acquiring white balance, the procedure continues to step S204. If the current operating state of the control apparatus 200 in step S203 is not acquiring white balance, the procedure continues to step S211.

In step S204, the preprocessor 110 goes into a state of standby of the calibration of the defective pixel correction. When the calibration of the defective pixel correction is in the state of standby, the controller 102 starts the imaging operation by the imager 106 in order to acquire white balance in the processor 210. The process then continues to step S205.

In step S205, the preprocessor 110 acquires the status information stored in the endoscope information memory 112. In step S206, the preprocessor 110 refers to the status information and determines whether or not the operation status of the current control apparatus 200 is the completion the white balance acquisition. As mentioned earlier, when the endoscope 100 receives the status signal from the system controller 202, the signal indicating that the white balance is currently being acquired, the controller 102 starts the imaging operation by the imager 106. The inner face of the white balance cap is colored white. If the white balance gain setting is appropriate, the white color of the white balance cap is correctly replicated by the white balance correction. If, however, the white balance gain setting is not appropriate, the color of the white balance cap is reddish or bluish due to white balance correction. The processor 210 calculates the white balance gain (white balance R gain, white balance B gain) so that the white color of the white balance cap becomes a predetermined reference white color. In this manner, the white balance is adjusted. After the white balance adjustment, the processor 210 notifies the system controller 202 that the white balance adjustment has been completed. In response to this, the system controller 202 transmits to the endoscope 100 a status signal indicating that the white balance acquisition has been completed. Based on this status signal, the preprocessor 110 makes the determination of step S206. If it is determined in step S206 that the operation status of the current control apparatus 200 is the completion of the white balance acquisition, the procedure continues to step S207. If it is determined in step S206 that the operation status of the current control apparatus 200 is not the completion of the white balance acquisition, the procedure returns to step S205.

In step S207, the preprocessor 110 acquires the status information stored in the endoscope information memory 112. In step S208, the preprocessor 110 refers to the status information and determines whether or not the operation status of the current control apparatus 200 is turning off the light source 208. When the white balance acquisition is completed, the system controller 202 turns off the light source 208. After turning off the light source 208, the system controller 202 transmits a status signal to the endoscope 100, the signal indicating that the light source 208 is currently turned off. Based on this status signal, the preprocessor 110 makes the determination in step S208. If it is determined in step S208 that the current operating state of the control apparatus 200 is turning off the light source, the procedure continues to step S209. If it is determined in step S208 that the current operating state of the control apparatus 200 is not turning off the light source, the procedure returns to step S207.

In step S209, the controller 102 initializes the imaging operation by the imager 106. In step S210, the preprocessor 110 calibrates, based on the imaging signal output from the imager 106, both the defective pixel correction and the black level correction.

The light source 208 is turned off by the control of the system controller 202. An imaging signal having a constant black level is therefore output in general from each pixel of the imager 106. If, however, a white defective pixel exists in the imager 106, only that white defective pixel outputs an imaging signal larger than the black level. The imaging signal of the white defective pixel is corrected by, for example, replacing it with the average value of the imaging signals of pixels surrounding the white defective pixel to be corrected. It is therefore necessary to specify the position of the white defective pixel. Here, the white defective pixel increases or decreases under the influence of changes in ambient temperature and deterioration over time. It is therefore desirable that the position of the white defective pixel be calibrated at an appropriate timing. In the present embodiment, this calibration is performed in a state in which the white balance cap is mounted and the light source 208 is turned off, that is, in a state where imaging in a dark place can be performed. The position of the white defective pixel is detected as the position of the pixel outputting, out of all the imaging signals output from the imager 106 as a result of dark place imaging, the imaging signal that is larger than the threshold value. The position of the detected white defective pixel is stored in the endoscope information memory 112.

If black level fluctuations, such as black floating or black sinking, are occurring in the imaging signal of the imager 106, the average value of the imaging signal does not reach the desired black level. The calibration for the black level correction is done in the present embodiment in the state in which the white balance cap is mounted and the light source 208 is turned off, i.e., in the state in which the imaging takes place in a dark place. The black level correction calibration is done by comparing the average value of the imaging signal output from the imager 106 as the result of the dark place imaging with the reference black level, and calculating the difference as the offset amount. The calculated offset amount is stored in the endoscope information memory 112. Note that the average value of the imaging signal is used for the black level correction calibration. It is therefore desirable that the black level correction be calibrated while the defective pixel correction is being performed. It is thus desirable that the process of step S210 be performed in the order: defective pixel correction calibration and then black level correction calibration.

Subsequent to the defective pixel correction calibration and black level correction calibration, the preprocessor 110 notifies the system controller 202 of the control apparatus 200 via the communication circuit 104 of the completion of the defective pixel correction calibration and black level correction calibration. In response to the notification, the system controller 202 turns on the light source 208.

In step S211, the controller 102 performs the imaging operation by the imager 106. In step S212, the preprocessor 110 preprocesses the imaging signal outputted from the imager 106. The preprocessing includes processes such as amplification of the imaging signal from the imager 106, A/D conversion, defective pixel correction, black level correction etc. In the defective pixel correction process, the preprocessor 110 reads the position of the defective pixel stored in the endoscope information memory 112 and interpolates the imaging signal at the position of the defective pixel using the imaging signal of the surrounding pixels. In the black level correction process, the preprocessor 110 reads the offset amount stored in the endoscope information memory 112, and adds/subtracts the offset amount to/from the imaging signal of each pixel. After completing the various preprocesses including the defective pixel correction and the black level correction, the procedure continues to step S213. Note that in step S212, a demosaicing process may be performed according to the imaging mode shown in FIG. 2.

In step S213, the preprocessor 110 transmits the pre-processed imaging signal to the control apparatus 200 via the communication circuit 104. Upon receiving the imaging signal via the communication circuit 204, the processor 210 performs image processing on the imaging signal according to the type of the endoscope 100 received in advance. The processor 210 then outputs the image data generated by the image processing to, for example, the monitor.

In step S214, the controller 102 determines whether or not to end the operation of the endoscope 100. It is determined that the operation of the endoscope 100 be ended when, for example, the endoscope 100 is removed from the control apparatus 200 or when an instruction is received from the control apparatus 200 to end the operation of the endoscope 100 due to a power-off operation or the like. When it is determined in step S214 that the operation of the endoscope 100 not be ended, the procedure continues to step S202. When it is determined in step S214 that the operation of the endoscope 100 be ended, the process of FIG. 3 ends.

As described above, in this embodiment, the preprocessor 110 of the endoscope 100 adaptively changes the contents of the pre-processing according to the operation mode of the control apparatus or the status signal indicating the operation state transmitted from the control apparatus 200. In this manner, the processor 210 of the control apparatus 200 does not need to be configured to be able to perform various processes according to the type of the imager 106 disposed in the endoscope 100. It is therefore possible to avoid an increase in circuit size of the processor 210.

In the above embodiment, an example is given in which the status signal is transmitted from the control apparatus 200. However, the status signal may also be generated inside the endoscope 100. For example, the operation unit 114 of the endoscope 100 may include a freeze button and a release button. A status signal indicating the timing at which the freeze button and the release button are operated may be input to the preprocessor 110, and the defective pixel correction calibration and black level correction calibration may be performed at the timing when this status signal is input. In this case, the preprocessor 110 itself functions as the status signal acquisition circuit. At the timing when these buttons are pushed by the user, the insertion part of the endoscope 100 is inserted into the subject, and it becomes unnecessary to perform imaging for the displaying at the monitor. Therefore, if the light source 208 is turned off at this timing, imaging may be performed in a dark place similarly to the case where the white balance cap is mounted.

Each process according to the above embodiment can also be stored as a program executable by a CPU or the like. They can also be stored in a storage medium of an external storage device such as a magnetic disk, an optical disk, a semiconductor memory, and then be distributed. The CPU or the like then reads the program stored in the storage medium of the external storage device, and the operation is controlled by the read program so that the above-described processing can be executed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An endoscope connected to a control apparatus, comprising:
an imager configured to image a subject to generate an imaging signal related to the subject;
a status signal acquisition circuit configured to receive a status signal indicating an operation status or an operation mode of the endoscope or of the control apparatus; and
a preprocessor configured to process the imaging signal according to the status signal received via the status signal acquisition circuit
wherein the status signal indicating the operation mode includes information indicating whether an imaging mode for the subject is a white light imaging mode or a special light imaging mode, and
the preprocessor performs different pixel interpolation processes onto the imaging signal according to whether the imaging mode indicated by the status signal is the white light imaging mode or the special light imaging mode.

2. The endoscope according to claim 1, wherein the status signal is transmitted from the control apparatus.

3. (canceled)

4. The endoscope according to claim 1, wherein the preprocessor performs linear interpolation onto the imaging signal when the status signal indicating that the imaging mode is the white light imaging mode has been received, and performs adaptive color plane interpolation onto the imaging signal when the signal indicating that the imaging mode is the special light imaging mode is received.

5. An endoscope connected to a control apparatus, comprising:
an imager configured to image a subject to generate an imaging signal related to the subject;
a status signal acquisition circuit configured to receive a status signal indicating an operation status or an operation mode of the endoscope or of the control apparatus; and
a preprocessor configured to process the imaging signal according to the status signal received via the status signal acquisition circuit,
wherein the status signal indicating the operation status includes both information indicating that the control apparatus has completed acquisition of white balance and information indicating that the control apparatus has completed turning off a light source, and
the preprocessor performs defective pixel correction calibration with respect to the imaging signal when both the status signal indicating that acquisition of the white balance gain has been completed and the status signal indicating that turning off the light source has been completed is received.

6. The endoscope according to claim 5, wherein the defective pixel correction calibration includes position detection of white defective pixels.

7. An endoscope connected to a control apparatus, comprising:
an imager configured to image a subject to generate an imaging signal related to the subject;
a status signal acquisition circuit configured to receive a status signal indicating an operation status or an operation mode of the endoscope or of the control apparatus; and
a preprocessor configured to process the imaging signal according to the status signal received via the status signal acquisition circuit,
wherein the status signal indicating the operation status includes both information indicating that the control apparatus is currently acquiring white balance and information indicating that the control apparatus has completed turning off a light source, and
the preprocessor performs black level correction calibration with respect to the imaging signal when both the status signal indicating that the white balance is currently being acquired and the status signal indicating that turning off the light source has been completed is received.
